(19) United States
(12) Patent Application Publication
(10) Pub. No.: US 2020/0046776 A1
(43) Pub. Date: Feb. 13, 2020

(54) STEM CELLS AND METHODS OF USING THEM

(71) Applicant: Animal Cell Therapies Inc., San Diego, CA (US)

(72) Inventors: Jiunn-chern Yeh, San Diego, CA (US); Kathryn J. Petrucci, La Jolla, CA (US)

(21) Appl. No.: 16/535,663

(22) Filed: Aug. 8, 2019

Related U.S. Application Data
(60) Provisional application No. 62/716,608, filed on Aug. 9, 2018.

(51) Int. Cl. A61K 35/32; A61K 38/17

(52) U.S. Cl. CPC A61K 35/32; A61K 38/1774

(57) ABSTRACT

This document relates to materials and methods involved in assessing and using stem cells. For example, materials and methods for determining if a stem cell (e.g., a mesenchymal stem cell) has an increased proliferation rate and/or an increased potential to differentiate into a bone cell are provided.

### CROSS-REFERENCE TO RELATED APPLICATIONS

This application claims the benefit of U.S. Provisional Application Ser. No. 62/716,608, filed Aug. 9, 2018. The disclosure of the prior application is considered part of (and is incorporated by reference in) the disclosure of this application.

### BACKGROUND

### 1. Technical Field

This document relates to materials and methods involved in assessing and using stem cells. For example, this document provides materials and methods for determining if a stem cell (e.g., a mesenchymal stem cell) has an increased proliferation rate and/or an increased potential to differentiate into a bone cell.

### 2. Background Information

Stem cells are undifferentiated cells having the ability to differentiate into a broad range of cell types. The importance of regulating the proliferation and differentiation of stem cells is well known. CD34 is widely accepted as a negative maker for mesenchymal stem cells (MSCs) (Dominici et al., Cytotherapy, 8:315-317 (2006); and Wagey and Short, J. Stem Cells Res Rev. & Rep., 1:1016 (2014)).

### SUMMARY

This document provides materials and methods for assessing and using stem cells. In some cases, a stem cell (e.g., a MSC) can be assessed to determine if it has increased potential to differentiate into a bone cell based, at least in part, on the expression level of cluster of differentiation 34 (CD34) polypeptides. For example, low initial expression of CD34 polypeptides can be effective to enhance differentiation of the stem cell to a bone cell. In some cases, a stem cell (e.g., a MSC) can be assessed to determine if it has an increased proliferation rate based, at least in part, on the expression level of CD34 polypeptides. For example, high initial expression of CD34 polypeptides can be effective to increase stem cell proliferation rates.

As described herein, a large fraction of freshly harvested and cultured canine umbilical cord tissue MSCs (UC-MSCs) were CD34 positive, and expressed differential levels of surface CD34 polypeptide. For example, cultured canine UC-MSCs initially (e.g., after one day) displayed differential levels of CD34 polypeptide expression, and the CD34 polypeptide expression diminished after a few days (e.g., about 2-3 days) in culture. Canine UC-MSCs having higher initial CD34 polypeptide expression had higher proliferation rate than the cells with lower CD34 expression. Canine UC-MSCs having lower initial CD34 polypeptide expression had higher osteocyte differentiation potential.

In general, one aspect of this document features methods for identifying a stem cell having increased proliferation rate. The methods can include, or consist essentially of, determining that a stem cell has high expression of CD34 polypeptides; and classifying the stem cell as having increased proliferation rate. The increased proliferation rate can be increased relative to a stem cell that has low expression of CD34 polypeptides. The increased proliferation rate can include about 1.36 or more doublings/day (e.g., about 1.39 ±0.03 doublings/day). The stem cell can be a canine stem cell. The stem cell can be a mesenchymal stem cell. The stem cell can have an increased proliferation rate after 2 passages in cell culture.

In another aspect, this document features methods for identifying a stem cell having increased potential to differentiate into a bone cell. The methods can include, or consist essentially of, determining that a stem cell has low expression of CD34 polypeptides; and classifying the stem cell as having increased potential to differentiate into a bone cell. The increased potential to differentiate into a bone cell can be increased relative to a stem cell that has high expression of CD34 polypeptides. The bone cell can be an osteocyte. The stem cell can be a canine stem cell. The stem cell can be a mesenchymal stem cell. The stem cell can have an increased potential to differentiate into a bone cell after 2 passages in cell culture.

In another aspect, this document features methods for treating a mammal having a bone disease. The methods can include, or consist essentially of, administering one or more stem cells having low expression of CD34 polypeptides to a mammal having a bone disease. The mammal can be a dog. The stem cell can be a canine stem cell. The stem cell can be a mesenchymal stem cell. The bone disease can be osteogenesis imperfecta or hypophosphatasia.

In another aspect, this document features methods for treating a mammal having a broken bone. The methods can include, or consist essentially of, administering one or more stem cells having low expression of CD34 polypeptides to a mammal having a broken bone. The mammal can be a dog. The stem cells can include canine stem cells. The stem cells can include mesenchymal stem cells. The broken bone can include a non-union fracture.

In another aspect, this document features methods for treating a mammal having an inflammatory disease. The methods can include, or consist essentially of, administering one or more stem cells having high expression of CD34 polypeptides to a mammal having an inflammatory disease. The mammal can be a dog. The stem cells can include canine stem cells. The stem cells can include mesenchymal stem cells. The inflammatory disease can be osteoarthritis.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows CD34 expression in canine UC-MSCs. Cells stained with anti-canine CD34 antibody were sorted according to their CD34 expression levels.

FIG. 2 shows CD34 sorted canine UC-MSCs from FIG. 1 that were re-run on flow cytometer to verify the expression intensity.

FIG. 3 shows that UC-MSCs with higher initial CD34 expression have greater proliferation potential.

FIG. 4 shows osteocyte differentiation of CD34 expressing UC-MSCs.

### DETAILED DESCRIPTION

This document provides materials and methods for assessing and using stem cells. In some cases, a stem cell (e.g., a MSC) can be assessed to determine if it has increased potential to differentiate into a bone cell based, at least in part, on the expression level of CD34 polypeptides. For example, low initial expression of CD34 polypeptides can be effective to enhance differentiation of the stem cell to a bone cell. In some cases, a stem cell (e.g., a MSC) can be assessed to determine if it has an increased proliferation rate based, at least in part, on the expression level of CD34 polypeptides. For example, high initial expression of CD34 polypeptides can be effective to increased stem cell proliferation rates. As used herein, the term “initial” as used with respect to CD34 polypeptide expression refers to the level of polypeptide expression determined in a stem cell that has not undergone any passaging in culture. For example, initial expression level of CD34 polypeptides can be assessed after about 1-3 days in culture.

A stem cell to be assessed as described herein can be obtained (e.g., harvested) from any appropriate source. In some cases, stem cells can be obtained (e.g., isolated) from a mammal. Examples of mammals from which stem cells can be obtained include, without limitation, a dog, a cat, and a horse. In some cases, stem cells can be obtained from a stem cell culture. In some cases, stem cells can be induced (e.g., from a somatic cell) in culture. In some cases, stem cells can be maintained, assessed, and/or expanded in culture. For example, MSCs can be obtained from a dog (e.g., canine stem cells), and can be maintained in culture for about 1 day to about 3 days prior to being assessed for CD34 polypeptide expression.

Any appropriate type of stem cell can be assessed as described herein. Examples of stem cells include, without limitation, MSCs (e.g., MSCs obtained from UC tissue, umbilical cord blood, adipose tissue, or bone marrow), neural stem cells (NSCs) (e.g., NSCs obtained from a brain tissue such as striatum), embryonic stem cells (ESCs), and induced pluripotent stem cells (iPSCs). For example, UC-MSCs can be assessed for increased proliferation rate and/or an increased potential to differentiate into a bone cell, based at least in part on the expression level of CD34 polypeptides.

Any appropriate method can be used to determine the expression level of CD34 polypeptides in a stem cell. For example, flow cytometry (e.g., fluorescence-activated cell sorting (FACS)) after labeling with an anti-CD34 antibody (e.g., a fluorescence-conjugated anti-CD34 antibody), immunohistochemistry (IHC) techniques, mass spectrometry techniques (e.g., proteomics-based mass spectrometry assays or targeted quantification-based mass spectrometry assays), western blotting techniques, and quantitative RT-PCR techniques, can be used to determine the expression level of CD34 polypeptides in a stem cell.

The term “expression” as used herein with respect to a level of CD34 polypeptides refers to any detectable level of CD34 polypeptides that is higher (e.g., about 50% higher) than a negative control (e.g., stained cells that do not express CD34 polypeptides and/or unstained control cells), but lower than a medium expression level of CD34 polypeptides. Any appropriate methods and/or techniques can be used to determine a level of CD34 polypeptides in a stem cell (e.g., a stem cell population). For example, a stem cell population can be contacted (e.g., stained) with a molecule that can bind to CD34 polypeptides (e.g., anti-CD34 antibodies) to identify CD34 positive stem cells, and the stem cell population can be sorted (e.g., using flow cytometry such as fluorescent activated cell sorting (FACS)) with gating to identify cells within a stem cell population that have “low expression,” “medium expression,” and “high expression” of CD34 polypeptides. In some cases, a stem cell population having a medium expression level of CD34 polypeptides can have from about 5 fold to about 10 fold higher CD34 polypeptide expression than a stem cell population having a low expression level of CD34 polypeptides. In some cases, a stem cell population having a high expression level of CD34 polypeptides can have from about 50 fold to about 100 fold higher CD34 polypeptide expression than a stem cell population having a low expression level of CD34 polypeptides. In cases where FACS is used to determine a level of CD34 polypeptides in a stem cell population, a mean fluorescence intensity (MFI) presented by geometric mean of each sample (e.g., a MFI analyzed by FlowJo software) can be used to determine an expression level of CD34 polypeptides in a stem cell population. For example, non-stained stem cells can have a geometric mean of about 1.07, isotype IgG stained stem cells can have geometric mean of about 1.13, and anti-CD34 stained stem cells, before sorting, can have a geometric mean of about 59.35. For example, after sorting, anti-CD34 stained stem cells having low expression of CD34 polypeptides can have a geometric mean of about 1.81, anti-CD34 stained stem cells having medium expression of CD34 polypeptides can have a geometric mean of about 12.96, and anti-CD34 stained stem cells having high expression of CD34 polypeptides can have a geometric mean of about 194.49.

The expression level of CD34 polypeptides in a stem cell can be used to determine if the stem cell has an increased proliferation rate and/or an increased potential to differentiate into a bone cell. In some cases, the expression level of CD34 polypeptides in a stem cell can be used to determine if the stem cell has an increased proliferation rate and/or an increased potential to differentiate into a bone cell as set forth in Table 1.

TABLE 1
CD34 expression in MSCs.

| CD34 | |
|---|---|
| expression level | stem cell phenotype |
| low | increased potential to differentiate into a bone cell |
| medium | moderately increased potential to differentiate into a |
| | bone cell (e.g., relative to stem cells having a high |
| | CD34 expression level) |
| medium | moderately increased proliferation (e.g., relative to |
| | stem cells having a low CD34 expression level) |
| high | increased proliferation |

MSCs can differentiate into many different types of cells, include, for example, osteoblasts, adipocytes, chondrocytes, myocytes, and/or neurons. An “increased potential to differentiate into a bone cell” with respect to a stem cell expressing CD34 refers to the ability of a stem cell to preferentially differentiate into a bone cell (e.g., rather than another cell type). In some cases, a stem cell having low initial expression of CD34 polypeptides as described herein can differentiate into a bone cell. For example, a UC-MSC having low initial expression of CD34 polypeptides can differentiate into an osteocyte. In some cases, a stem cell having low initial expression of CD34 polypeptides as described herein can have increased levels of calcium (e.g., as compared to a stem cell having medium initial expression of CD34 polypeptides and/or a stem cell having high initial expression of CD34 polypeptides). For example, a UC-MSC having low initial expression of CD34 polypeptides can have increased levels of calcium (e.g., as compared to a UC-MSC having medium initial expression of CD34 polypeptides and/or a UC-MSC having high initial expression of CD34 polypeptides).

A stem cell having low expression of CD34 polypeptides can have the potential to differentiate into any appropriate type of bone cell. Examples of bone cells include, without limitation, osteocytes, osteoclasts, and osteoblasts. Any appropriate method can be used to determine whether or not a stem cell has differentiated into a bone cell. For example, quantitative osteocyte differentiation assays (e.g., calcium quantification with Alizarin Red S) and/or detection of bone differentiation factors can be used to determine whether or not a stem cell has differentiated into a bone cell.

A stem cell (e.g., a CD34 negative canine UC-MSC) typically proliferates at a rate of about 1.31±0.23 doublings per day (e.g., at passage 2). However, proliferation rates can be affected by culture conditions (e.g., culture medium and/or culture substrates used for cells to attach) and/or passage number. An “increased proliferation rate” with respect to a stem cell expressing CD34 refers to any rate of proliferation that is faster than a proliferation rate of a stem cell that does not express CD34. In some cases, a proliferation rate can be measured as cell doublings per day (cell doublings/days in culture). For example, a proliferation rate can be measured as using the following formula: cell doublings=3.32 (log [number of cells harvested]−log [number of cells seeded]). In some cases, a stem cell having high initial expression of CD34 polypeptides as described herein can have a proliferation rate of about 1.36 or more doublings/day. For example, a UC-MSC having high initial expression of CD34 polypeptides can have a proliferation rate of about 1.39±0.03 doublings/day. In some cases, a stem cell having medium initial expression of CD34 polypeptides as described herein can have a proliferation rate of about 1.30 doublings/day to about 1.35 doublings/day. For example, a UC-MSC having medium initial expression of CD34 polypeptides can have a proliferation rate of about 1.33±0.02 doublings/day. In some cases, a stem cell having low initial expression of CD34 polypeptides as described herein can have a proliferation rate of about 1.29 or fewer doublings/day. For example, a UC-MSC having low initial expression of CD34 polypeptides can have a proliferation rate of about 1.25±0.03 doublings/day. Any appropriate method can be used to assess the proliferation rate of a stem cell.

For example, cell counting (e.g., manual counting of cells under microscopy observation), dye (e.g., trypan blue) exclusion to count only viable cells, flow cytometry (e.g., for a biomarker such as Ki67), and/or metabolic activity assays (e.g., MTT assays) can be used to assess the proliferation rate of a stem cell.

When a stem cell (e.g., a MSC) is identified as having an increased potential to differentiate into a bone cell based, at least in part, on having low expression of CD34 polypeptides as described herein, the stem cell can be used to treat a mammal having a bone disease and/or having a broken bone (e.g., by administering one or more stem cells having low initial expression of CD34 polypeptides to a mammal having a bone disease and/or having a broken bone). A bone disease can include bones that break easily. A bone disease can include abnormal development of bones. Examples of bone diseases that can be treated as described herein include, without limitation, osteogenesis imperfecta and hypophosphatasia. A broken bone can be any bone within a mammal's body. A broken bone can be any type of broken bone. In some cases, a broken bone can be a non-union fracture. In some cases, a bone disease and/or a broken bone that can be treated as described herein can be described elsewhere (see, e.g., Beyth et al., 2011 Br Med Bull. 00:199-210). In some cases, a population of MSCs where at least about 75% (e.g., about 75%, about 80%, about 85%, about 90%, about 95%, about 97%, or about 99%) of the MSCs have an increased potential to differentiate into a bone cell based, at least in part, on having low expression of CD34 polypeptides as described herein, can be used to treat a mammal having a bone disease and/or having a broken bone. For example, one or more stem cells having low expression of CD34 polypeptides as described herein can be administered to a mammal having a bone disease and/or having a broken bone. In some cases, one or more stem cells having low expression of CD34 polypeptides can be administered intravenously to a mammal having a bone disease and/or having a broken bone. In some cases, one or more stem cells having low expression of CD34 polypeptides can be injected into one or more sites of a mammal having a bone disease and/or having a broken bone that are affected by the bone disease and/or the broken bone.

When a stem cell (e.g., a MSC) is identified as having an increased proliferation rate based, at least in part, on having high expression of CD34 polypeptides as described herein, the stem cell can be used to treat a mammal having an inflammatory disease (e.g., by administering one or more stem cells having low initial expression of CD34 polypeptides to a mammal having an inflammatory disease). An inflammatory disease can be any type of inflammatory disease. In some cases, an inflammatory disease can be osteoarthritis. In some cases, a population of MSCs where at least about 75% (e.g., about 75%, about 80%, about 85%, about 90%, about 95%, about 97%, or about 99%) of the MSCs have an increased proliferation rate based, at least in part, on having high expression of CD34 polypeptides as described herein, can be used to treat a mammal having an inflammatory disease. For example, one or more stem cells having high expression of CD34 polypeptides as described herein can be administered intravenously to a mammal having an inflammatory disease. In some cases, one or more stem cells having low expression of CD34 polypeptides can be injected into one or more sites of a mammal having an inflammatory disease that are affected by the inflammatory disease.

The invention will be further described in the following examples, which do not limit the scope of the invention described in the claims.

### EXAMPLES

Example 1

### CD34 Polypeptide Expression in Canine UC-MSCs

CD34 is widely accepted as a negative maker for mesenchymal stem cells (MSCs). Cultured canine MSCs isolated from different tissue sources were also reported in the literatures to be CD34 negative. However, we found that a large fraction of canine umbilical cord tissue-derived MSCs were CD34 positive after they were harvested from the tissues and attached to the culture dishes. To investigate whether there is a difference in canine MSCs expressing different levels of CD34, the cells were fractionated into CD34 high-, medium- and low-expressers based on their expression levels using a cell sorter. The proliferation and osteocyte differentiation potentials of these cells were evaluated.

Harvesting of Canine UC-MSCs from Umbilical Cord Tissues:

Canine UC-MSCs were harvested from freshly donated umbilical cord tissues. All tissue processing was performed using sterile technique. The umbilical cord tissues were washed and separated from the placentas and the vasculatures. The cords were washed two additional times in pre-filled sterile petri dishes containing Hank's balanced salt solution with antibiotics/antimycotic reagents. The fluid was squeezed out from the cords using gentle pressure of sterile scissors. The cords were placed in an empty sterile petri dish and weighed on an analytical balance. The cords were minced and digested with enzyme solution containing collagenase/hyaluronidase and digested at 37° C. rocking platform incubator for tissue digestion. At the end of the digestion, the tissue was removed from the rocking platform incubator and placed under the biological safety cabinet. The solution was pipetted gently to disperse the cells from the tissues and was slowly filtered through 100 μm cell strainers. One million cells were seeded in a 10-cm culture dish and ensure that the processed cells were able to grow to confluency and no gross contamination was observed. The remaining cells were centrifuged, the supernatant removed and re-suspended in freezing medium (DMEM/10% FCS/10% DMSO). Frozen cells (passage 0) were stored at ˜80° C. liquid nitrogen tank.

### Culturing of Canine UC-MSCs:

Passage 0 cells were thawed and seeded in 15-cm culture dishes (10′ cells per dish; total 6 dishes). Cells were harvested from the dishes two days after seeding with TrypLE Express (Life Technologies) and neutralized with DMEM/10% FCS. After centrifugation, cells were re-suspended in DMEM/10% FCS, and the cells numbers and viability were determined by trypan blue exclusion method on a hemocytomerter.

### Cell Sorting:

Three sets of cells, unstained control, control isotype IgG stained and anti-canine CD34 stained, were prepared for cell sorting. For non-stained control, 10⁵ cells were centrifuged and re-suspended in 100 μl sterile antibody staining solution (PBS with 10 mg/ml BSA). For isotype IgG control, 10⁵ cells were centrifuged and re-suspended in 100 μl sterile antibody staining solution. Five microliters of PE-conjugated mouse IgG1 kappa isotype control antibody (eBioscience Cat #12-4714-42) were added to the cells. For CD34 staining, about 5×10\hat{ } 6 cells were re-suspended in 1 ml of antibody staining solution. Fifty microliters of PE-conjugated anti-canine CD34 antibody (eBioscience Cat #12-0340-42) were added to the cells. Cells were incubated at 4° C. with occasional gentle agitation.

After one hour, cells were centrifuged at x350 g at 4° C. for 5 minutes. After removing the supernatant, cold PBS (0.4 ml each for non-stained and isotype IgG control, and 4 ml for anti-CD34 stained cells) were added to the tubes to re-suspend/wash the cells.

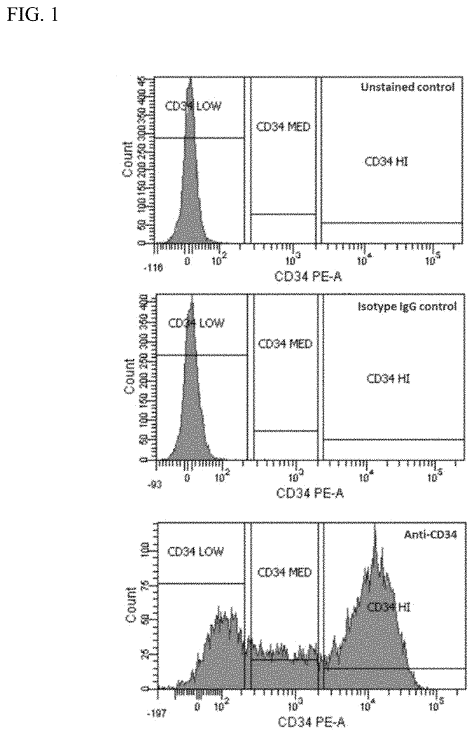

Cells were centrifuged at x350 g at 4° C. for 5 minutes. After removing the supernatant, the cells were re-suspended in sterile cold sorting buffer (PBS/1 mM EDTA/1% FCS). Propidium iodine was added to the cells (1 μg per 400 μl of cell solution) right before the sorting to identify the dead cells. Cells were sorted on a BD Aria II cell sorter. Unstained and isotype control IgG stained cells were used to set up the gate for CD34 positive cells. CD34-stained cells were then sorted based on the CD34 expression level into CD34 low (CD34-L; cells with CD34 low staining intensity, slightly higher than the negative control), CD34 medium (CD34-M; cells with intermediate CD34 expression) and CD34 high (CD34-H; cells with high CD34 expression) (FIG. 1)

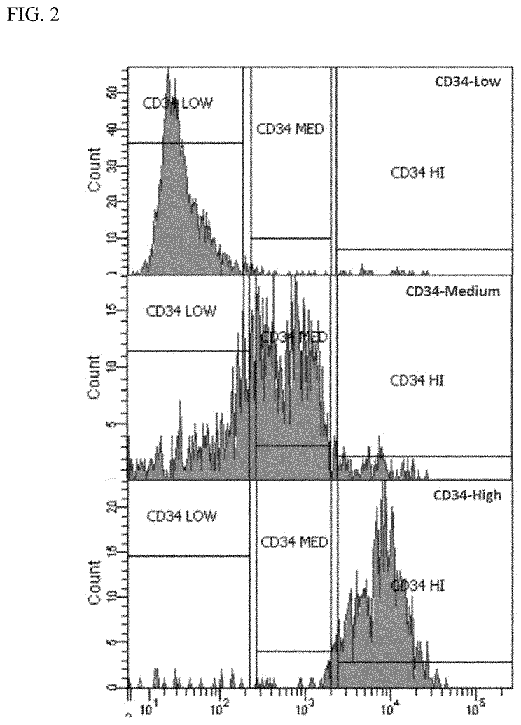

Regular culture media was used to collect the sorted cells. Sorted cells were re-run on cell sorter to verify the expression intensity (FIG. 2)

### Proliferation Rate of CD34-Sorted Cells:

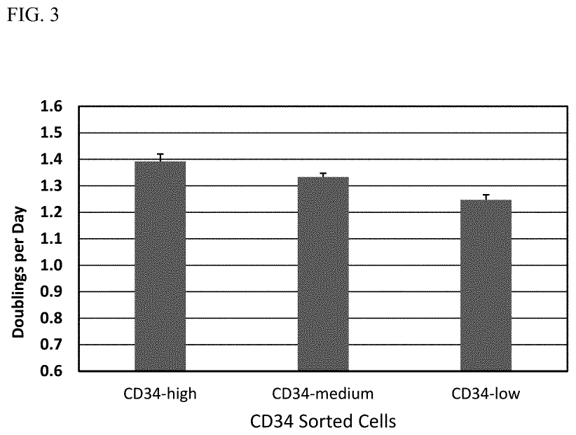

The sorted cells were centrifuged and re-suspended in DMEM/10% FCS to determine the cell number and viability. 2.5×10⁴ cells from each sorted group were seeded in 35 mm culture dishes. After cells reached ˜80% confluency (four days after seeding), cells (passage 1) were harvested TrypLE Express as described above to determine the cell number and viability. 2.5×10⁴ passage 1 cells from each group were seeded in 35 mm culture dishes and cultured for three days. Cells (passage 2) were harvested to determine the cell number and viability. The doubling number of cells at each passage was calculated using the following formula: cell doublings=3.32 (log [number of cells harvested]−log [number of cells seeded]). Proliferation rate was defined as cell doublings per day (cell doublings days in culture). No significant difference was observed in the proliferation rate in passage one sorted cells. However, there was a significant different in the proliferation rate at passage 2 (FIG. 3). The doublings per day for each group (n=4 for each group) was 1.39±0.03 for CD34-high, 1.33±0.02 for CD34-medium and 1.25±0.03 for CD34-low. The differences between each group were all statistically significant (student t-test, p=0.0153 for high vs medium; p=0.0004 for high vs low; p=0.0017 for medium vs low).

### Osteocyte Differentiation:

The sorted cells at passage 2 were seeded at 5×10⁴ per well in 12-well plates. When cells reach 70 to 80% (2 to 3 days), the culture medium was replaced with osteocyte differentiation medium (Life Technologies, StemPro Osteogenesis Differentiation Kit, Cat.# A10072-01). Control cells which continued to grow in regular culture medium served as the “un-differentiated control”. Cells were fixed on the differentiation induction day to serve as the “day 0 control”. The media were replaced every two to three days. After two to three weeks of osteogenic induction, cells were fixed with 4% paraformaldehyde in PBS.

The fixed cells were stained with 2% Alizarin Red S (ARS; prepared in dH2O, pH 4.1) for 10 minutes. After washing three times with dH2O, the bound ARS was extracted with 10% cetylpryrdinium chloride (prepared in PBS). The extracted ARS was quantified on a microplate reader at OD 405 nm by comparing with results with a standard curve established using defined concentrations of ARS (from 8 μg/ml to 1000 μg/ml).

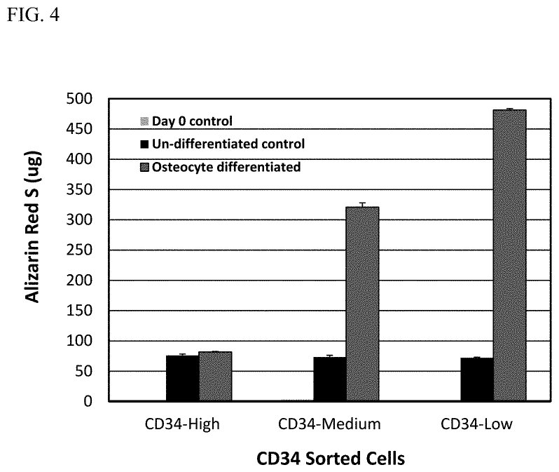

An inverse relationship was observed between the initial CD34 expression levels and their osteocyte differentiation potency (FIG. 4). The cells with initial CD34 expression had lower osteocyte differentiation potency. The extracted ARS for each group was as following: for day 0 controls, CD34-high is 2.39±0.11 μg, CD34-medium is 3.02±0.11 μg and CD34-low is 2.55±0.11 μg; for non-differentiated controls, CD34-high is 76.06±2.21 μg, CD34-medium is 73.33±2.98 μg and CD34-low is 72.16±0.88 μg; for osteogenic differentiated cells, CD34-high is 81.84±0.88 μg, CD34-medium is 320.91±7.29 μg and CD34-low is 481.38±2.43 μg.

These results demonstrated that the expression level of CD34 polypeptides in a stem cell can be used to determine if the stem cell has an increased potential to differentiate into a bone cell and/or an increased proliferation rate based.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a stem cell having increased potential to differentiate into a bone cell, wherein said method comprises:
(a) determining that said stem cell has low expression of CD34 polypeptides; and
(b) classifying said stem cell as having increased potential to differentiate into a bone cell.

2. The method of claim 1, wherein said increased potential to differentiate into a bone cell is increased relative to a stem cell that has high expression of CD34 polypeptides.

3. The method of claim 1, wherein said bone cell is an osteocyte.

4. The method of claim 1, wherein said stem cell is a canine stem cell.

5. The method of claim 1, wherein said stem cell is a mesenchymal stem cell.

6. The method of claim 1, wherein said stem cell has an increased potential to differentiate into a bone cell after 2 passages in cell culture.

7. A method for treating a mammal having a bone disease, said method comprising administering one or more stem cells having low expression of CD34 polypeptides to said mammal.

8. The method of claim 7, wherein said mammal is a dog.

9. The method of claim 7, wherein said one or more stem cells comprise canine stem cells.

10. The method of claim 7, wherein said one or more stem cells comprise mesenchymal stem cells.

11. The method of claim 7, wherein said bone disease is osteogenesis imperfecta or hypophosphatasia.

12. A method for treating a mammal having a broken bone, said method comprising administering one or more stem cells having low expression of CD34 polypeptides to said mammal.

13. The method of claim 12, wherein said mammal is a dog.

14. The method of claim 12, wherein said one or more stem cells comprise canine stem cells.

15. The method of claim 12, wherein said one or more stem cells comprise mesenchymal stem cells.

16. The method of claim 12, wherein said broken bone comprises a non-union fracture.
